(19) United States
(12) Patent Application Publication
(10) Pub. No.: US 2014/0064454 A1
(43) Pub. Date: Mar. 6, 2014

(54) X-RAY SYSTEM AND METHOD WITH DIGITAL IMAGE ACQUISITION USING A PHOTOVOLTAIC DEVICE

(71) Applicants: Christopher Alden Hammond, Farmington Hills, MI (US); Ping Xue, Pewaukee, WI (US); Diego Fernando Freire Muñoz, Chicago, IL (US); Amanda Lynn Pratt, Hartland, WI (US); Robert Carl Minnich, Cortland, OH (US); Gregory Donald Schumacher-Novak, Milwaukee, WI (US); Michael Lee Spohn, Waukesha, WI (US)

(72) Inventors: Christopher Alden Hammond, Farmington Hills, MI (US); Ping Xue, Pewaukee, WI (US); Diego Fernando Freire Muñoz, Chicago, IL (US); Amanda Lynn Pratt, Hartland, WI (US); Robert Carl Minnich, Cortland, OH (US); Gregory Donald Schumacher-Novak, Milwaukee, WI (US); Michael Lee Spohn, Waukesha, WI (US)

(73) Assignee: GENERAL ELECTRIC COMPANY, Schenectady, NY (US)

(21) Appl. No.: 13/597,090

(22) Filed: Aug. 28, 2012

(51) Int. Cl. H05G 1/38; G01T 1/24; G01T 1/16

(52) U.S. Cl. 378/96; 250/336.1; 250/370.04

(57) ABSTRACT

An X-ray imaging system is provided. The X-ray imaging system includes an X-ray radiation source. The X-ray imaging system also includes a source controller coupled to the source and configured to command emission of X-rays for image exposures. The X-ray imaging system further includes a digital X-ray detector configured to acquire X-ray image data without communication from the source controller, wherein the digital X-ray detector includes a photovoltaic device, and the digital X-ray detector is configured to determine one or more of a beginning, end, or duration of an image exposure via the photovoltaic device.

### BACKGROUND

The subject matter disclosed herein relates to X-ray imaging systems and more particularly to X-ray imaging systems using digital detectors having photovoltaic devices.

The advent of digital X-ray detectors has brought enhanced workflow and high image quality to medical imaging. However, many of the earlier radiographic imaging systems employ conventional X-ray imaging using film as the X-ray detection media. In order to obtain images from these systems, the imaging medium must be transported and processed after each exposure, resulting in a time delay in obtaining the desired images. Digital radiography provides an alternative that allows the acquisition of image data and reconstructed images on the spot for quicker viewing and diagnosis, and allows for images to be readily stored and transmitted to consulting and referring physicians and specialists. However, the cost of replacing the earlier conventional radiographic imaging systems with digital radiographic imaging systems may be imposing to a hospital or tertiary care medical center. Hence, there is a need to retrofit the earlier radiographic imaging systems for digital radiography in a cost effective manner involving as few components of the systems as possible.

### BRIEF DESCRIPTION

In accordance with a first embodiment, an X-ray imaging system is provided. The X-ray imaging system includes an X-ray radiation source. The X-ray imaging system also includes a source controller coupled to the source and configured to command emission of X-rays for image exposures. The X-ray imaging system further includes a digital X-ray detector configured to acquire X-ray image data without communication from the source controller, wherein the digital X-ray detector includes a photovoltaic device, and the digital X-ray detector is configured to determine one or more of a beginning, end, or duration of an image exposure via the photovoltaic device.

In accordance with a second embodiment, a digital X-ray detector is provided. The detector includes circuitry configured to acquire X-ray image data without communication from an X-ray source controller. The detector also includes a photovoltaic device, wherein the circuitry is configured to determine one or more of a beginning, end, or duration of an image exposure via the photovoltaic device.

In accordance with a third embodiment, an X-ray imaging method is provided. The method includes monitoring a voltage or current level of a photovoltaic device of a digital X-ray detector. The method also includes commanding an X-ray radiation source to perform an X-ray exposure via a source controller coupled to the source, the source controller not being in communication with the X-ray detector. The method further includes determining one or more of a beginning, end, or duration of the X-ray exposure based on the voltage level of the photovoltaic device.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a perspective view of a fixed X-ray system, equipped in accordance with aspects of the present technique;

FIG. 2 is a perspective view of a mobile X-ray system, equipped in accordance with aspects of the present technique;

FIG. 3 is a diagrammatical overview of the X-ray system in FIGS. 1 and 2;

FIG. 4 is a diagrammatical representation of functional components in a detector of the system of FIGS. 1-3;

FIG. 5 is an exploded perspective view of an embodiment of a detector assembly having a photovoltaic device;

FIG. 6 is a bottom schematic view of an embodiment of a photovoltaic device (e.g., a single solar cell) disposed on a detector array;

FIG. 7 is a bottom schematic view of an embodiment of a photovoltaic device (e.g., multiple solar cells) disposed on a detector array;

FIG. 8 is a flow diagram illustrating an embodiment of a method for determining a beginning, end, and duration of an exposure; and

FIG. 9 is a flow diagram illustrating an embodiment of a method for monitoring a beginning and end of an exposure.

### DETAILED DESCRIPTION OF THE INVENTION

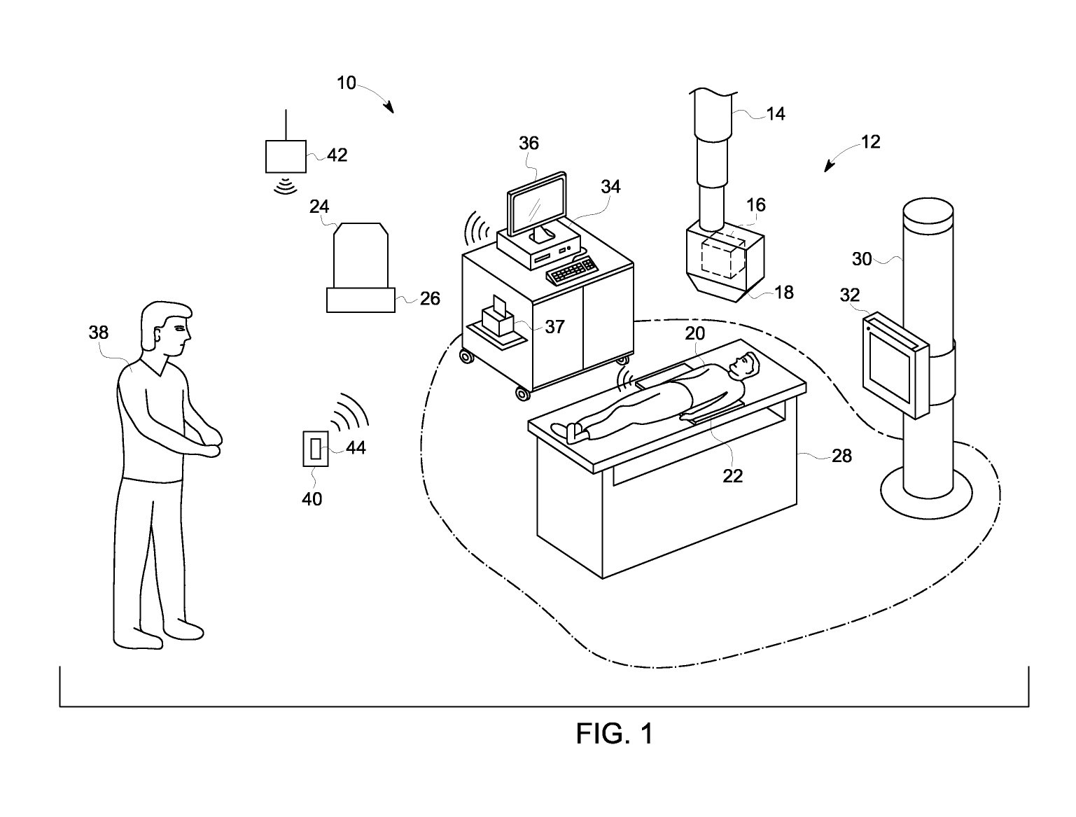

Referring generally to FIG. 1, an X-ray system is represented, referenced generally by reference numeral 10. In the illustrated embodiment, the X-ray system 10, as adapted, is a digital X-ray system. The X-ray system 10 is designed both to acquire image data and to process the image data for display in accordance with the present technique. Throughout the following discussion, however, while basic and background information is provided on the digital X-ray system used in medical diagnostic applications, it should be born in mind that aspects of the present techniques may be applied to digital detectors, including X-ray detectors, used in different settings (e.g., projection X-ray, computed tomography imaging, tomosynthesis imaging, etc.) and for different purposes (e.g., parcel, baggage, vehicle and part inspection, etc.).

In the embodiment illustrated in FIG. 1, the X-ray system 10 includes an imaging system 12. The imaging system 12 may be a conventional analog imaging system, retrofitted for digital image data acquisition and processing as described below. In one embodiment, the imaging system 12 may be a stationary system disposed in a fixed X-ray imaging room, such as that generally depicted in and described below with respect to FIG. 1. It will be appreciated, however, that the presently disclosed techniques may also be employed with other imaging systems, including mobile X-ray units and systems in other embodiments. The imaging system 12 includes an overhead tube support arm 14 for positioning a radiation source 16, such as an X-ray tube, and a collimator 18 with respect to a patient 20 and a detector 22. The detector 22 includes a digital X-ray detector. In some embodiments, the detector 22 may be selected from a plurality of detectors 22, represented by detector 24, from a dock 26 (e.g., charging dock). Each detector 22 of the plurality of detectors 22 may be labeled and designed for a particular type of imaging (e.g., fluoroscopic and radiographic imaging). The detector 22 is configured to acquire X-ray image data without communication from a controller of the X-ray radiation source 16. In other words, the detector 22 operates without communication of timing signals from the controller of the source 16 as to an X-ray exposure. Thus, the detector 22 is without a priori knowledge of the beginning and ending times of an exposure or the duration of the exposure. As a result, the detector 22 may include a photovoltaic device to enable the detector 22 to determine the beginning, end, and/or duration of an image exposure as described in greater detail below. Upon determining the end and/or beginning of the image exposure, the detector may begin and/or end sampling image data from the image exposure. Also, the detector 22 is configured to combine multiple imaging frames that include imaging data to generate X-ray images. In addition, the detector 22 is configured to at least partially process X-ray image data.

In one embodiment, the imaging system 12 may be used in concert with one or both of a patient table 28 and a wall stand 30 to facilitate image acquisition. Particularly, the table 28 and the wall stand 30 may be configured to receive detector 22. For instance, detector 22 may be placed on an upper, lower or intermediate surface of the table 28, and the patient 20 (more specifically, an anatomy of interest of the patient 20) may be positioned on the table 28 between the detector 22 and the radiation source 16. Also, the wall stand 30 may include a receiving structure 32 also adapted to receive the detector 22, and the patient 20 may be positioned adjacent the wall stand 30 to enable the image data to be acquired via the detector 22. The receiving structure 32 may be moved vertically along the wall stand 30.

Also depicted in FIG. 1, the imaging system 12 includes a workstation 34, display 36, and printer 37. In one embodiment, the workstation 34 may include or provide the functionality of the imaging system 12 such that a user 38, by interacting with the workstation 34 may control operation of the source 16 and detector 22. In other embodiments, the functions of the imaging system 12 may be decentralized, such that some functions of the imaging system 12 are performed at the workstation 34 (e.g., controlling operation of the source 16), while other functions (e.g., controlling operation of the detector 22) are performed by another component of the X-ray system 10, such as a portable detector control device 40. The portable detector control device 40 may include a personal digital assistant (PDA), palmtop computer, laptop computer, smart telephone, tablet computer, or any suitable general purpose or dedicated portable interface device. The portable detector control device 40 is configured to be held by the user 38 and to communicate wirelessly with the detector 22. It is noted that the detector 22 and portable detector control device 40 may utilize any suitable wireless communication protocol, such as an IEEE 802.15.4 protocol, an ultra wideband (UWB) communication standard, a Bluetooth communication standard, or any IEEE 802.11 communication standard. Alternatively, the portable detector control device 40 may be configured to be tethered or detachably tethered to the detector 22 to communicate via a wired connection.

The portable detector control device 40 is also configured to communicate instructions (e.g., detector operating mode) to the detector 22 for the acquisition of X-ray image data. In turn, the detector 22 is configured to prepare for an X-ray exposure in response to instructions from the portable detector control device 40, and to transmit a detector ready signal to the device 40 indicating that the detector 22 is prepared to receive the X-ray exposure. The device 40 may also be configured to communicate patient information or X-ray technique information to the detector 22. Similar to the detector 22, the device 40 may be without communication from the controller of the X-ray source 16. Further, the portable detector control device 40 is configured to receive X-ray image data from the detector 22 for processing and image reconstruction. Indeed, both the detector 22 and the portable detector control device 40 are configured to at least partially process the X-ray image data. However, in certain embodiments, the detector 22 and/or the portable detector control device 40 are configured to fully process the X-ray image data. Also, the detector 22 and/or the device 40 is configured to generate a DICOM compliant data file based upon the X-ray image data, patient information, and other information. Further, the detector 22 and/or the device 40 is configured to wirelessly transmit (or via a wired connection) processed X-ray image data (e.g., partially or fully processed X-ray image data) to an institution image review and storage system over a network 42. The institution image review and storage system may include a hospital information system (HIS), a radiology information system (RIS), and/or picture archiving communication system (PACS). In some embodiments, the institution image review and storage system may process the X-ray image data. In one embodiment, the workstation 34 may be configured to function as a server of instructions and/or content on a network 42 of the medical facility. The detector 22 and/or device 40 are also configured to transmit, via a wired or wireless connection, processed X-ray images to the printer 37 to generate a copy of the image.

The portable detector control device 40 includes a user-viewable screen 44 and is configured to display patient data and reconstructed X-ray images based upon X-ray image data on the screen 44. The screen 44 may include a touch-screen and/or input device (e.g., keyboard) configured to input data (e.g., patient data) and/or commands (e.g., to the detector). For example, the device 40 may be used to input patient information and other imaging related information (e.g., type of source 16, imaging parameters, etc.) to form a DICOM image header. In one embodiment, the patient information may be transferred from a patient database via a wireless or wired connection from the network or the workstation 34 to the device 40. The detector 22 and/or device may incorporate the information for the image header with the X-ray image to generate the DICOM compliant data file. Also, the device 40 may be used to navigate X-ray images displayed on the screen 44. Further, the device 40 may be used to modify the X-ray images, for example, by adding position markers (e.g., “L”/“R”for left and right, respectively) onto the image. In one embodiment, metal markers may be placed on the detector 22 to generate position markers.

In one embodiment, the imaging system 12 may be a stationary system disposed in a fixed X-ray imaging room, such as that generally depicted in and described above with respect to FIG. 1. It will be appreciated, however, that the presently disclosed techniques may also be employed with other imaging systems, including mobile X-ray units and systems, in other embodiments.

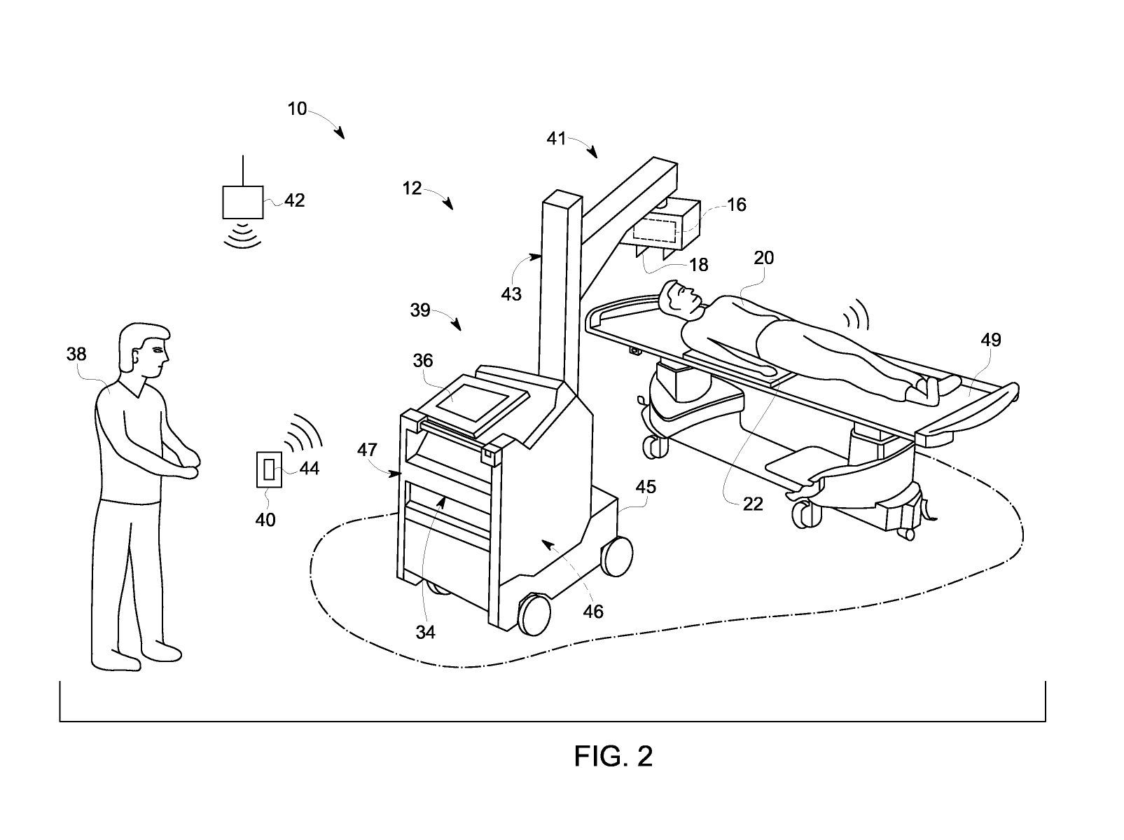

For instance, as illustrated in the X-ray system of FIG. 2, the imaging system 12 may be moved to a patient recovery room, an emergency room, a surgical room, or any other space to enable imaging of the patient 20 without requiring transport of the patient 20 to a dedicated (i.e., fixed) X-ray imaging room. The imaging system 12 includes a mobile X-ray base station 39 and detector 22. Similar to above, the imaging system 12 may be a conventional analog imaging system, retrofitted for digital image data acquisition and processing. In one embodiment, a support arm 41 may be vertically moved along a support column 43 to facilitate positioning of the radiation source 16 and collimator 18 with respect to the patient 20. Further, one or both of the support arm 41 and support column 43 may also be configured to allow rotation of the radiation source 16 about an axis. Further, the X-ray base station 39 has a wheeled base 45 for movement of the station 39. Systems electronic circuitry 46 with a base unit 47 both provides and controls power to the X-ray source 16 and the wheeled base 45 in the imaging system 12. The base unit 47 also has the operator workstation 34 and display 36 that enables the user 38 to operate the X-ray system 10. The operator workstation 34 may include buttons, switches, or the like to facilitate operation of the X-ray source 16. Similar to the X-ray system 10 in FIG. 1, the system 10 includes the portable control device 40. The detector 22 and portable control device 40 are as described above. In the X-ray system, the patient 20 may be located on a bed 49 (or gurney, table or any other support) between the X-ray source 16 and the detector 22 and subjected to X-rays that pass through the patient 20 and are received by the detector 22.

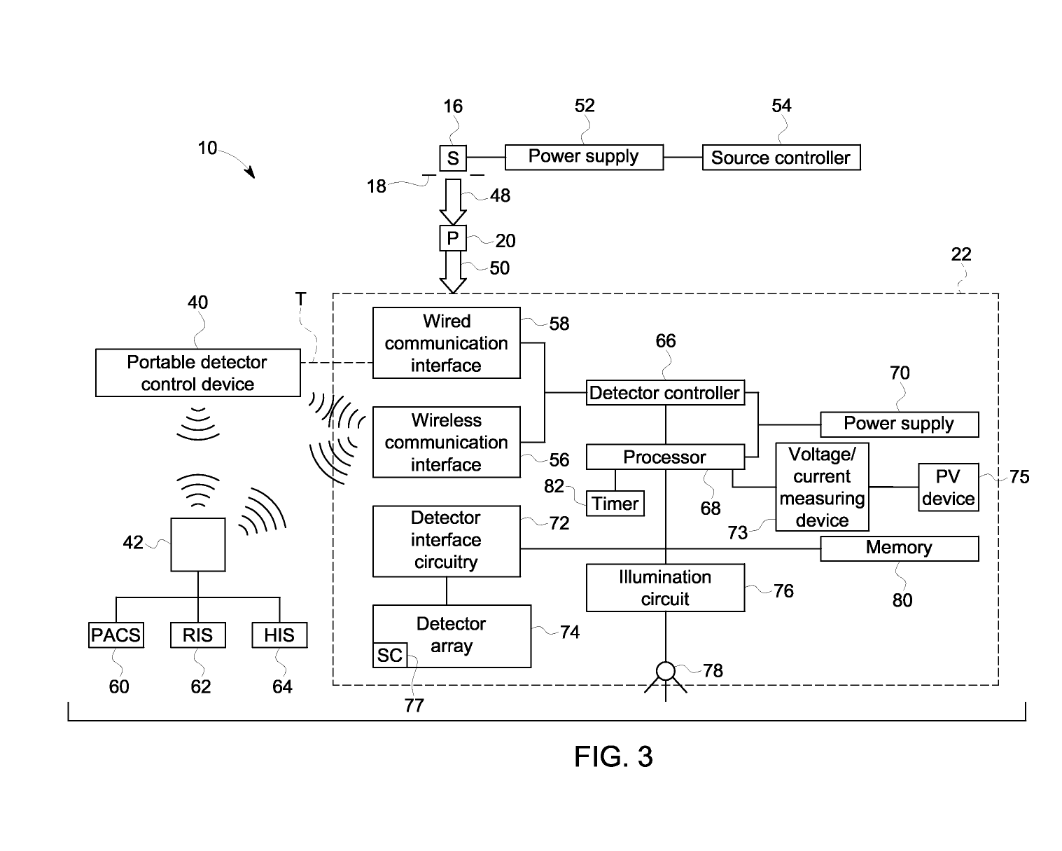

FIG. 3 is a diagrammatical overview of the X-ray system 10 in FIGS. 1 and 2 illustrating the components of the system 10 in more detail. The imaging system 10 includes the X-ray radiation source 16 positioned adjacent to a collimator 18. Collimator 18 permits a stream of radiation 48 to pass into a region in which a subject 20, such as a human patient 20, is positioned. A portion of the radiation 50 passes through or around the subject 20 and impacts the digital X-ray detector 22. As described more fully below, detector 22 converts the X-ray photons received on its surface to lower energy photons, and subsequently to electric signals which are acquired and processed to reconstruct an image of the features within the subject 20.

The source 16 is coupled to a power supply 52 which furnishes power for examination sequences. The source 16 and power supply 52 are coupled to a source controller 54 configured to command X-ray emission of X-rays for image exposures. As mentioned above, the detector 22 is configured to acquire X-ray image data without communication from the source controller 54. Also, the detector 22 is responsive to the portable detector control device 40 configured to communicate instructions the detector 22 for acquisition of the X-ray image data. In addition, the portable detector control device 40 is configured to receive the X-ray image data from the detector 22 for processing and imaging reconstruction.

The detector 22 includes a wireless communication interface 56 for wireless communication with the device 40, as well as a wired communication interface 58, for communicating with the device 40 when it is tethered to the detector 22. The detector 22 and/or the device 40 may also be in communication with the institution image review and storage system over the network 42 via a wired or wireless connection. As mentioned above, the institution image review and storage system may include PACS 60, RIS 62, and HIS 64. In certain embodiments, the detector 22 may also communicate with components of the imaging system 12 such as the operator workstation 34 via a wired or wireless connection. It is noted that the wireless communication interface 56 may utilize any suitable wireless communication protocol, such as an ultra wideband (UWB) communication standard, a Bluetooth communication standard, or any 802.11 communication standard. Moreover, detector 22 is coupled to a detector controller 66 which coordinates the control of the various detector functions. For example, detector controller 66 may execute various signal processing and filtration functions, such as for initial adjustment of dynamic ranges, interleaving of digital image data, and so forth. The detector controller 66 is responsive to signals from the device 40. The detector controller 66 is linked to a processor 68. The processor 68, the detector controller 66, and all of the circuitry receive power from a power supply 70. The power supply 70 may include one or more batteries. Also, the processor 68 is linked to detector interface circuitry 72.

The detector 22 converts X-ray photons received on its surface to lower energy photons such as light or optical photons (e.g., via a scintillator 77). The detector 22 includes a detector array 74 (e.g., imaging panel) that includes an array of photodetectors to convert the light photons to electrical signals. In certain embodiments, the detector array 74 also includes the scintillator 77. These electrical signals are converted to digital values by the detector interface circuitry 72 which provides the values to the processor 68 to be converted to imaging data and sent to the device 40 to reconstruct an image of the features within the subject 20. In one embodiment, the detector 22 may at least partially process or fully process the imaging data. Alternatively, the imaging data may be sent from the detector 22 to a server to process the imaging data.

The processor 68 is also linked to a voltage/current measuring device 73. The voltage/current measuring device 73 is coupled to a photovoltaic device 75. The photovoltaic device 75, via the photovoltaic effect or photoconductive effect (if reverse biased), generates a voltage or current in response to optical photons and/or X-rays received, e.g., from the scintillator, on a surface of the device 75. Thus, the voltage or current of the photovoltaic device 75 may be monitored to determine the beginning, end, and/or duration of an image exposure. In certain embodiments, the voltage or current of the photovoltaic device may be monitored to control an auto-exposure control. The voltage measuring device 73 measures the voltage or current generated by the photovoltaic device 75. The voltage/current measuring device 73 may include any type of data collecting or measuring device such as an analog-to-digital converter, field-programmable gate array, and so forth. The photovoltaic device 75 may include one or more solar panels as described in greater detail below. In certain embodiments, the solar panels may include semiconductor materials reactive to X-ray or visible light spectrum. In other embodiments, the device 75 may include a semiconductor device arranged to serve a similar function as the solar panels (i.e., collect the optical photons and/or X-rays to enable determining the beginning, end, and/or duration of an image exposure).

The processor 68 is further linked to an illumination circuit 76. The detector controller 66, in response to a signal received from the device 40, may send a signal to the processor 68 to signal the illumination circuit 76 to illuminate a light 78 to indicate the detector 22 is prepared to receive an X-ray exposure in response to the signal. Indeed, in response to a signal from the device 40, the detector 22 may be turned on or awoken from an idle state. Alternatively, the detector 22 may be turned on directly or awoken from an idle state by the user (e.g., pressing an on/off button located on the detector 22). As another alternative, the detector 22 may be awoken from an idle or lower-powered state upon detecting the beginning of an exposure via the photovoltaic device 75.

Further, the processor is linked to a memory 80. The memory 80 may store various configuration parameters, calibration files, and detector identification data. In addition, the memory 80 may store patient information received from the device 40 to be combined with the image data to generate a DICOM compliant data file. Further, the memory 80 may store sampled data gathered during the imaging mode as well as X-ray images. As mentioned above, in some embodiments, the device 40 may conduct the image processing and incorporate a DICOM header to generate a DICOM compliant data file. Still further, the processor 68 is linked to a timer 82 to monitor times for multiple purposes such as determining the duration of an exposure.

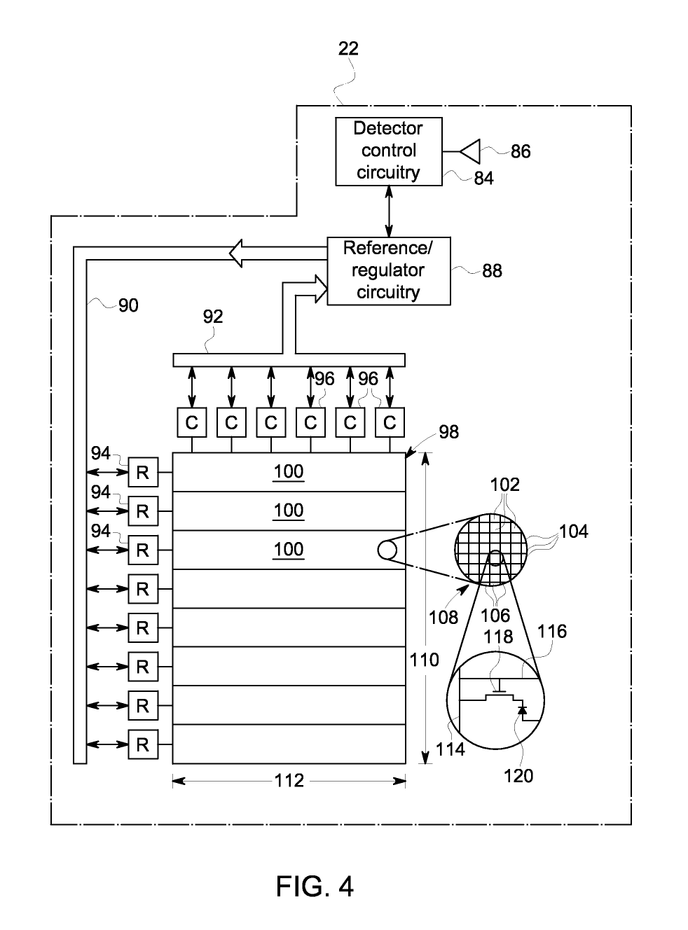

FIG. 4 is a diagrammatical representation of functional components of digital detector 22. As illustrated, detector control circuitry 84 receives DC power from a power source, represented generally at reference numeral 86. Detector control circuitry 84 is configured to originate timing and control commands for row and column electronics used to acquire image data during data acquisition phases of operation of the system. Circuitry 84 therefore transmits power and control signals to reference/regulator circuitry 88, and receives digital image pixel data from circuitry 88.

In a present embodiment, detector 22 consists of a scintillator that converts X-ray photons received on the detector surface during examinations to lower energy (light) photons. An array of photodetectors then converts the light photons to electrical signals which are representative of the number of photons or the intensity of radiation impacting individual pixel regions or picture elements of the detector surface. Readout electronics convert the resulting analog signals to digital values that can be processed, stored, and displayed, such as on device 40 following reconstruction of the image. In a present form, the array of photodetectors is formed of amorphous silicon. The array of photodetectors or discrete picture elements is organized in rows and columns, with each discrete picture element consisting of a photodiode and a thin film transistor. The cathode of each diode is connected to the source of the transistor, and the anodes of all diodes are connected to a negative bias voltage. The gates of the transistors in each row are connected together and the row electrodes are connected to the scanning electronics as described below. The drains of the transistors in a column are connected together and the electrode of each column is connected to an individual channel of the readout electronics. As described in greater detail below, the detector control circuitry 84 is configured to sample data from the discrete picture elements during receipt of X-ray radiation in response to the photovoltaic device 75 detecting the beginning of the exposure and to cease sampling upon detecting the end of the exposure.

Turning back to the embodiment illustrated in FIG. 4, by way of example, a row bus 90 includes a plurality of conductors for enabling readout from various rows of the detector 22, as well as for disabling rows and applying a charge compensation voltage to selected rows, where desired. A column bus 92 includes additional conductors for commanding readout from the columns while the rows are sequentially enabled. Row bus 90 is coupled to a series of row drivers 94, each of which commands enabling of a series of rows in the detector 22. Similarly, readout electronics 96 are coupled to column bus 92 for commanding readout of all columns of the detector.

In the illustrated embodiment, row drivers 94 and readout electronics 96 are coupled to a detector panel 98 which may be subdivided into a plurality of sections 100. Each section 100 is coupled to one of the row drivers 94, and includes a number of rows. Similarly, each column driver 96 is coupled to a series of columns. The photodiode and thin film transistor arrangement mentioned above thereby define a series of pixels or discrete picture elements 102 which are arranged in rows 104 and columns 106. The rows and columns define an image matrix 108, having a height 110 and a width 112.

As also illustrated in FIG. 4, each picture element 102 is generally defined at a row and column crossing, at which a column electrode 114 crosses a row electrode 116. As mentioned above, a thin film transistor 118 is provided at each crossing location for each picture element, as is a photodiode 120. As each row is enabled by row drivers 94, signals from each photodiode 120 may be accessed via readout electronics 96, and converted to digital signals for subsequent processing and image reconstruction. Thus, an entire row of picture elements 102 in the array is controlled simultaneously when the scan line attached to the gates of all the transistors 118 of picture elements 102 on that row is activated. Consequently, each of the picture elements 102 in that particular row is connected to a data line, through a switch, which is used by the readout electronics to restore the charge to the photodiode 120.

It should be noted that in certain systems, as the charge is restored to all the picture elements 102 in a row simultaneously by each of the associated dedicated readout channels, the readout electronics is converting the measurements from the previous row from an analog voltage to a digital value. Furthermore, the readout electronics may transfer the digital values from rows previous to the acquisition subsystem, which will perform some processing prior to displaying a diagnostic image on a monitor or writing it to film.

The circuitry used to enable the rows may be referred to in a present context as row enable or field effect transistor (FET) circuitry based upon the use of field effect transistors for such enablement (row driving). The FETs associated with the row enable circuitry described above are placed in an “on” or conducting state for enabling the rows, and are turned “off” or placed in a non-conducting state when the rows are not enabled for readout. Despite such language, it should be noted that the particular circuit components used for the row drivers and column readout electronics may vary, and the present invention is not limited to the use of FETs or any particular circuit components.

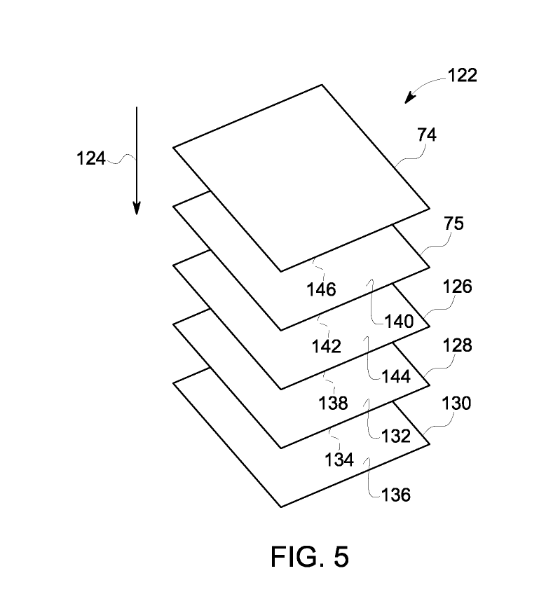

FIG. 5 depicts an exploded view of a detector assembly 122 for the detector 22. It should be noted that the following detector assembly 122 may include other components not described (e.g., an outer cover or sleeve, handle, etc.). Arrow 124 indicates a direction of the X-ray path relative to the detector assembly 122. The detector includes the detector array 74, photovoltaic device 75, backscattered X-ray blocking layer 126, panel support 128, and motherboard 130. The panel support 128 supports the motherboard 130 and the detector array 74. In addition, the panel support 128 mechanically isolates the imaging components of the detector array 74 from the electronics of the motherboard 130. Generally, the panel support 128 may be formed of a metal, a metal alloy, a plastic, a composite material, or a combination of the above material. In one embodiment, the panel support 128 may be substantially formed of a carbon fiber reinforced plastic material or a graphite fiber-epoxy composite. In another embodiment, the panel support 128 may be substantially formed of composite materials in combination with a foam core in a laminated sandwich construction so as to provide a lightweight yet stiff assembly to serve as the panel support 128.

The panel support 128 includes a surface 132 (e.g., front or top surface) and a surface 134 (e.g., rear or bottom surface) disposed opposite from each other. The backscattered X-ray blocking layer 126 and motherboard 130 are disposed on or coupled to surfaces 132, 134, respectively, of the panel support 128. In particular, surface 134 of the panel support 128 is disposed on or coupled to surface 136 (e.g., front or top surface) of the motherboard 130. Also, surface 138 (e.g., rear or bottom surface) of the backscattered X-ray blocking layer 126 is disposed on or coupled to surface 132 of the panel support 128. The motherboard 130 includes a circuit board and electronics including row drivers 94 and readout electronics 96 to acquire signals from the detector array 74. The backscattered X-ray blocking layer 126 may include lead to minimize X-ray backscattering. X-rays may pass through the detector array 74 and reflect back off whatever is found behind the detector array 74 such as the electronics or panel support 128. The reflected X-rays may be detected by the scintillator layer, converted to light, and detected by the photosensitive layer in the detector elements. The backscattered X-ray blocking layer 126 may absorb the X-rays passing through the detector array 74 and any backscattered X-rays.

The photovoltaic device 75 includes a surface 140 (e.g., front or top surface) and a surface 142 (e.g., rear or bottom surface) disposed opposite from each other. The detector array 74 and backscattered X-ray blocking layer 126 are disposed on or coupled to surfaces 140, 142, respectively, of the photovoltaic device 75. In particular, surface 142 of the photovoltaic device 75 is disposed on or coupled to the surface 144 (e.g., front or top surface) of the backscattered X-ray blocking layer 126. Also, surface 146 (e.g., rear or bottom surface) of the detector array 74 is disposed on or coupled to surface 140 of the photovoltaic device 75. The photovoltaic device 75 is disposed between the detector array 74 and the backscattered X-ray blocking layer 126 so that the photovoltaic device 75 does not obstruct X-ray detection by the detector array 74.

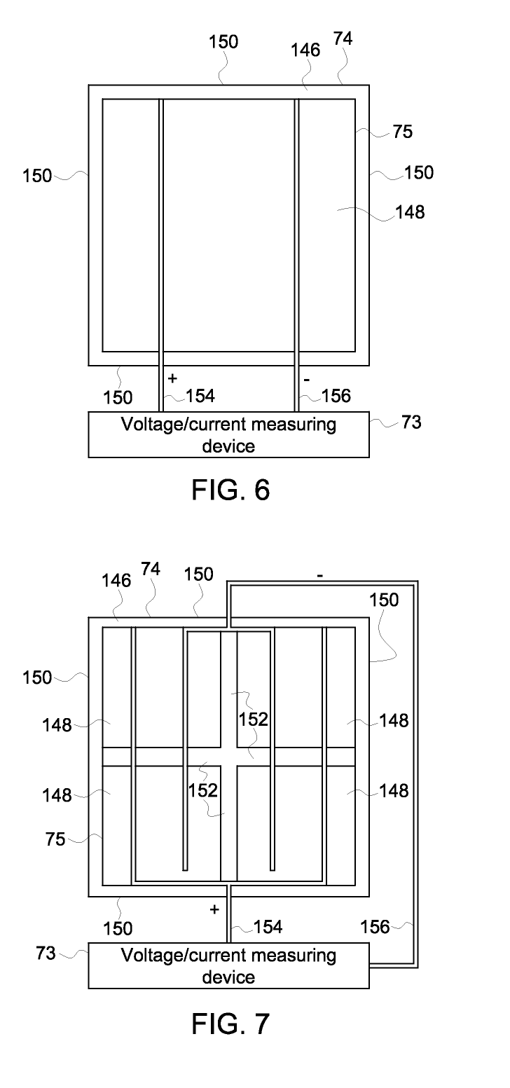

FIGS. 6 and 7 illustrate embodiments of the photovoltaic device 75 disposed on the photodetector array 74 and coupled to the voltage/current measuring device 73. The photovoltaic device 75 may include solar panels or any other type of photovoltaic device 75 (e.g., utilizing the photovoltaic effect or photoconductive effect (if reverse biased)). The photovoltaic device 75 may include one or more solar panels. As depicted in FIG. 6, the photovoltaic device 75 includes a single solar panel 148. As depicted in FIG. 7, the photovoltaic device 75 includes four solar panels 148. It should be noted, the photovoltaic device 75 may include any number of solar panels 148. The solar panels 148 in FIGS. 6 and 7 may include monocrystalline solar cells (e.g., monocrystalline silicon), polycrystalline solar cells (e.g., polycrystalline silicon), flexible solar cells (e.g., amorphous silicon), or any combination thereof, or any other type solar panel or cell. Further, the solar cells may include a variety of materials such cadmium telluride, copper indium gallium selenide, or gallium arsenide, or any other material. In certain embodiments, the solar panels may include semiconductor materials reactive to X-ray or visible light spectrum. In other embodiments, the device 75 may include a semiconductor device arranged to serve a similar function as the solar panels (i.e., collect the optical photons and/or X-rays to enable determining the beginning, end, and/or duration of an image exposure).

For illustrative purposes, the photovoltaic device 75 of FIGS. 6 and 7 does not cover the entire surface 146 of the detector array 74. However, in one embodiment, the photovoltaic device 75 will cover or extend across the entire surface 146 of the detector array 74 to edges 150 of the array 74. In embodiments with multiple solar cells 148 such as FIG. 6, there may be gaps 152 (e.g., of approximately 1 cm or less) between the solar cells 148. However, the area of the gaps 152 is substantially small (e.g., less than approximately 1 percent) relative to the surface area of the photovoltaic device 75 extending across the array 74. Thus, the photovoltaic device 75 extends substantially across the entire surface 146 of the array 74 in such embodiments. In other embodiments, the photovoltaic device 75 may extend across less than the entire surface 146 of the array 74. Indeed, the photovoltaic device 75 may extend across as little as a single pixel of the array 74. In certain embodiments, the photovoltaic device 75 may include the solar panels 148 in handful of locations or discontinuously scattered on the surface 146 of the array 74.

As depicted in FIGS. 6 and 7, the voltage/current measuring device 73 is coupled to the photovoltaic device 75 via lines or leads 154 and 156, positive and negative leads, respectively, to measure any voltage or current generated by the device 75. As depicted in FIG. 7, the leads 154, 156 branch off to couple to the individual solar cells 148. The voltage/current measuring device 73 may include any type of data collecting or measuring device such as an analog-to-digital converter, field-programmable gate array, and so forth.

As mentioned above, the detector 22 is without communication from the source controller 54 and, thus, is without a priori knowledge of the beginning and ending times of an exposure. In one embodiment, the detector 22 is configured to automatically determine or detect the beginning, end, and/or duration of the exposure utilizing the photovoltaic device 75 without communication from the source controller 54 and/or detector control device 40.

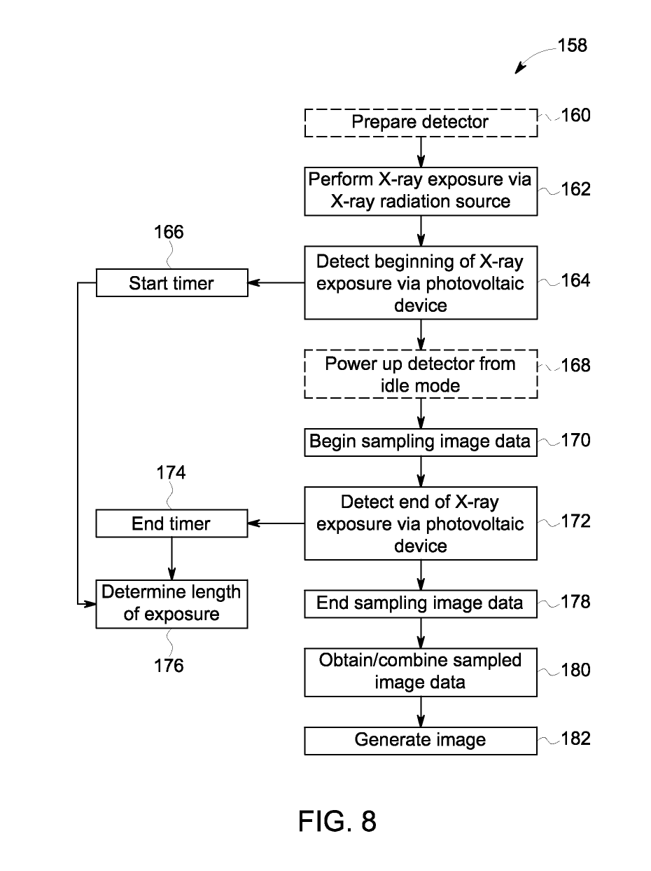

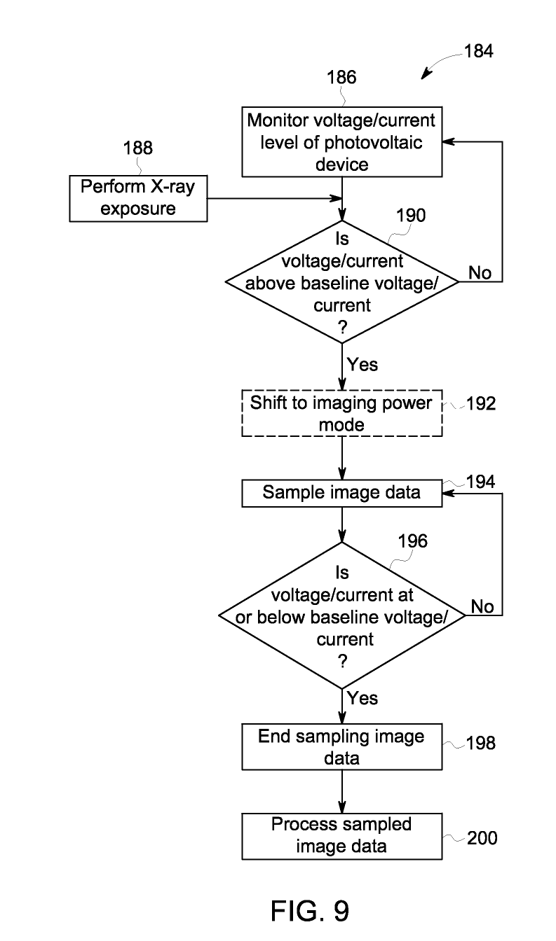

FIGS. 8 and 9 describe various embodiments of methods employing the photovoltaic device 75 to determine the beginning, end, and/or duration of an image exposure. FIG. 8 illustrates a method 158 for determining the beginning, end, and duration of an exposure. The method 158 may include preparing the detector 22 for the X-ray exposure (block 160). In certain embodiments, the user commands a detector preparation signal from the device 40 to the detector 22. Alternatively, the user may press a button on the detector 22 to begin preparation. Once the detector 22 receives the command to prepare from the device 40 or button, the detector 22 prepares for the acquisition of X-ray image data. For example, the detector 22 may switch from an idle mode to imaging power mode and begin scrubbing (i.e., preparing and refreshing the detector circuitry) the panel of the detector 22 to equilibrate the panel. After scrubbing, the detector 22 reads or acquires one or more offset frames prior to exposure. In certain embodiments, the offset frames may be acquired after the X-ray exposure. After preparation, the detector 22 sends to the device 40 the detector ready signal. In one embodiment, the detector 22 may also provide a visible indication (e.g., flashing light) or an audio indication to indicate the detector 22 is ready. In another embodiment, the detector control device 40 may provide a visible indication and/or audio indication. The user then commands the X-ray radiation source 16 to perform an X-ray exposure via the source controller 54 coupled to the source 16 (block 162). Upon beginning the exposure, the detector 22 converts incident radiation from the source 16 into optical photons, and the photovoltaic device 75 receives the optical photons and/or X-rays and generates a voltage or current in response to the received optical photons and/or X-rays. The generated voltage (e.g., in the range of approximately 1-20 millivolts) or current is measured by the voltage/current measuring device 73 and this enables the processor 68 to determine or detect the beginning of the X-ray exposure (block 164). In certain embodiments, the voltage or current signal collected from the photovoltaic device 75 may be amplified.

Upon detecting the beginning of the exposure, the processor 68 sends a signal to the timer 82 to start timing (block 166) the duration or length of the exposure. Also, in certain embodiments, upon detecting the beginning of the exposure, if the detector 22 is in an idle or low power mode prior to and during the beginning of the exposure, the detector 22 switches from the idle mode to imaging power mode (block 168).

Further, upon detecting the beginning of the exposure, the detector 22 begins sampling image data from during the exposure (block 170). During the exposure, the voltage/current measuring device 73 continues to monitor the voltage or current of the photovoltaic device 75. Once the generated voltage or current returns to pre-exposure levels, this enables the processor 68 to determine or detect the end of the exposure (block 172). Upon detecting the end of the exposure, the processor 68 sends a signal to the timer 82 to end or stop timing (block 174) the duration or length of the exposure. Upon stopping the timer 82, the processor 68 determines the duration or length of the exposure (block 176).

Also, upon detecting the end of the exposure, the detector 22 ends sampling of image data obtained during the exposure (block 178). In certain embodiments, the device 40 at least partially processes the X-ray image data. In some embodiments, the device 40 completely processes the X-ray image data. Alternatively, the device 40 acquires completely processed X-ray image data from the detector 22. In other embodiments, neither the detector 22 nor the device 40 completely process the X-ray image data, but send the X-ray image data to the institution image review and storage system for subsequent processing. In either case, to obtain an X-ray image, the sampled X-ray image data is obtained and/or combined (block 180) from one or more imaging frames. The combined data may be further processed (e.g., offset-corrected) prior to generating an X-ray image (block 182).

FIG. 9 illustrates a method 184 illustrating a method for determining a beginning and end of an X-ray exposure. The method 184 includes monitoring a voltage or current level or signal of the photovoltaic device 75 of the detector 22 via the voltage/current measuring device (block 186). At any time while monitoring the photovoltaic device 75, an X-ray exposure may be performed (block 188). The detector 22 (e.g., processor 68) continuously compares the voltage or current level obtained from the photovoltaic device to a baseline voltage or current (e.g., threshold) to determine if the obtained voltage or current level is above the baseline voltage or current (block 190). In some embodiments, the baseline voltage or current level may be zero. In other embodiments, the baseline voltage or current level may be set above zero to take into account any background voltage or current levels. If the obtained voltage or current level from the photovoltaic device 75 is not above the baseline voltage or current, this indicates an X-ray exposure has not begun and the detector 22 continues to monitor the voltage or current level of the photovoltaic device 75 (block 186). If the obtained voltage or current level from the photovoltaic device 75 is above the baseline voltage or current, this indicates an X-ray exposure has begun and, if the detector 22 is in an idle or low power mode prior to and during the beginning of the exposure, the detector 22 switches from the idle mode to imaging power mode (block 192). In addition, the obtained voltage or current level is above the baseline voltage or current, the detector 22 begins sampling image data from during the exposure (block 194).

Upon the determining the beginning of the X-ray exposure, the detector 22 continues to monitor the voltage or current level of the photovoltaic device 75 and compares the voltage or current level obtained from the photovoltaic device 75 to the baseline voltage or current to determine if the obtained voltage or current level has returned to or fallen below the baseline voltage or current (block 196). If the obtained voltage or current level from the photovoltaic device 75 has not returned to the baseline voltage or current, this indicates that the X-ray exposure is still occurring and the detector 22 continues to sample X-ray image data (block 194). If the obtained voltage or current level from the photovoltaic device 75 does return to the baseline voltage or current, this indicates the X-ray exposure has ended and the detector 22 ends sampling image data from during the exposure (block 198). The sampled X-ray image data may then be processed (block 200) as described above.

Technical effects of the disclosed embodiments include providing systems and methods to allow for the retrofitting of conventional X-ray systems by replacing cassettes with a digital X-ray detector. In retrofitting the X-ray systems, the digital X-ray detector 22 does not communicate with the X-ray imaging system 12. Since the detector 22 does not communicate with the X-ray imaging system 12, the detector 22 lacks data indicating the timing signals for an X-ray exposure. Thus, the detector 22 utilizes the photovoltaic device 75 to monitor the beginning, end, and/or duration of the X-ray exposure.

This written description uses examples to disclose the present subject matter, including the best mode, and also to enable any person skilled in the art to practice the present approaches, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An X-ray imaging system comprising:
an X-ray radiation source;
a source controller coupled to the source and configured to command emission of X-rays for image exposures; and
a digital X-ray detector configured to acquire X-ray image data without communication from the source controller, wherein the digital X-ray detector comprises a photovoltaic device, and the digital X-ray detector is configured to determine one or more of a beginning, end, or duration of an image exposure via the photvoltaic device.

2. The X-ray imaging system of claim 1, wherein the photovoltaic device comprises at least one solar cell.

3. The X-ray imaging system of claim 2, wherein the at least one solar cell comprises a monocrystalline solar cell, polycrystalline solar cell, flexible solar cell, cadmium telluride solar cell, copper indium gallium selenide solar cell, or gallium arsenide solar cell, or a combination thereof

4. The X-ray imaging system of claim 1, wherein the detector comprises an imaging panel and a panel support, and the photovoltaic device is disposed between the imaging panel and the panel support.

5. The X-ray imaging system of claim 4, wherein the photovoltaic device extends substantially across an entire surface of the imaging panel facing the photovoltaic device.

6. The X-ray imaging system of claim 1, wherein the photovoltaic device is configured to receive optical photons or X-rays and generate a voltage or current in response to the received optical photons or X-rays.

7. The X-ray imaging system of claim 6, wherein the detector comprises a voltage/current measuring device coupled to the photovoltaic device, and the voltage/current measuring device is configured to measure the voltage or current generated by the photovoltaic device in response to the received optical photons or X-rays.

8. The X-ray imaging system of claim 7, wherein the detector is configured both to detect the beginning of the image exposure and to begin sampling image data upon the voltage or current exceeding a baseline threshold.

9. The X-ray imaging system of claim 8, wherein the detector is configured both to detect the end of the image exposure and to end sampling image data upon the voltage or current returning to the baseline threshold.

10. The X-ray imaging system of claim 1, wherein the detector is configured to determine the beginning, end, and duration of the image exposure via the photovoltaic device.

11. A digital X-ray detector comprising:
circuitry configured to acquire X-ray image data without communication from an X-ray source controller; and
a photovoltaic device, wherein the circuitry is configured to determine one or more of a beginning, end, or duration of an image exposure via the photovoltaic device.

12. The digital X-ray detector of claim 11, wherein the photovoltaic device is configured to receive optical photons or X-rays and generate a voltage or current in response to the received optical photons or X-rays.

13. The digital X-ray detector of claim 12, wherein the detector comprises a voltage/current measuring device coupled to the photovoltaic device, and the voltage measuring device is configured to measure the voltage or current generated by the photovoltaic device in response to the received optical photons or X-rays.

14. The digial X-ray detector of claim 13, wherein the circuitry is configured both to detect the beginning of the image exposure and to begin sampling image data upon the voltage or current exceeding a baseline threshold.

15. The digital X-ray detector of claim 14, wherein the circuitry is configured both to detect the end of the image exposure and to end sampling image data upon the voltage or current returning to the baseline threshold.

16. The digital X-ray detector of claim 11, wherein the circuitry is configured to determine the beginning, end, and duration of the image exposure via the photovoltaic device.

17. The digital X-ray detector of claim 11, wherein the photovoltaic device comprises at least one solar cell.

18. The digital X-ray detector of claim 17, wherein the at least one solar cell comprises a monocrystalline solar cell, polycrystalline solar cell, flexible solar cell, cadmium telluride solar cell, copper indium gallium selenide solar cell, or gallium arsenide solar cell,or a combination thereof

19. The digital X-ray detector of claim 11, wherein the detector comprises an imaging panel and a panel support, and the photovoltaic device is disposed between the imaging panel and the panel support.

20. The digital X-ray detector of claim 19, wherein the photovoltaic device extends substantially across an entire surface of the imaging panel facing the photovoltaic device.

21. An X-ray imaging method comprising:
monitoring a voltage or current level of a photovoltaic device of a digital X-ray detector;
commanding an X-ray radiation source to perform an X-ray exposure via a source controller coupled to the source, the source controller not being in communication with the X-ray detector; and
determining one or more of a beginning, end, or duration of the X-ray exposure based on the voltage or current level of the photovoltaic device.

22. The method of claim 21, comprising, if the voltage or current level exceeds a baseline threshold, detecting the beginning of the X-ray exposure and starting sampling of image data.

23. The method of claim 22, comprising, if the voltage or current level returns to the baseline threshold, detecting the end of the X-ray exposure and ending sampling of image data.
